(19) United States
(12) Patent Application Publication
(10) Pub. No.: US 2021/0063384 A1
(43) Pub. Date: Mar. 4, 2021

(54) METHOD OF ASSESSING CIRCULATING TUMOR CELLS AND METHOD OF USING DATA OBTAINED THEREFROM

(71) Applicant: Chang Gung University, Taoyuan City (TW)

(72) Inventors: MIN-HSIEN WU, Taoyuan City (TW); CHIA-JUNG LIAO, Taoyuan City (TW)

(21) Appl. No.: 16/724,132

(22) Filed: Dec. 20, 2019

(30) Foreign Application Priority Data
Aug. 30, 2019 (TW) 108131164

(51) Int. Cl. G01N 33/50; C12N 5/09; C12Q 1/6806; G01N 33/574

(52) U.S. Cl. CPC G01N 33/5094; C12N 5/0693; C12N 2513/00; G01N 33/574; C12Q 1/6806

(57) ABSTRACT

Methods of assessing circulating tumor cells (CTCs) and methods of using the data obtained from assessing CTCs are provided. The methods employ negative selection and 3D cell culture techniques to isolate and culture CTCs. Those CTCs then can be used in different bioassays or evaluations, such as to determine tumor morphology, monitor tumor status, predict prognosis, provide treatment suggestion, and assess treatment effectiveness.

### CROSS-REFERENCE TO RELATED DISCLOSURE AND CLAIM OF PRIORITY

The present invention is related to a published disclosure entitled as “The Integration of a Three-Dimensional Spheroid Cell Culture Operation in a Circulating Tumor Cell (CTC) Isolation and Purification Process” in the journal of “Cancers” by MDPI, presented publicly as earliest as Jun. 6, 2019. The above mentioned published disclosure is made by or obtained directly or indirectly from the named inventor(s) or joint inventor(s) of the present invention. Entire disclosure of the above-mentioned published disclosure is incorporated by reference herein. The present invention also claims benefit of Taiwan Application No. 108131164, filed on Aug. 30, 2019, the disclosure of which is incorporated by reference herein.

### 1. TECHNICAL FIELD

At least one embodiment of the present invention relates to a method of assessing circulating tumor cells (CTCs) and a method of using the data obtained from assessing CTCs. More particularly, the methods are based on a technique to sort nucleated cells as well as determine cell counts to evaluate the characteristics and future development of tumors.

### 2. DESCRIPTION OF THE RELATED ART

Circulating tumor cells (CTCs) are one of the materials frequently used in the field of liquid biopsy. The liquid biopsy technique can be used to determine CTCs count in a blood sample, and to diagnose diseases, monitor disease progression, assess treatment effectiveness, as well as predict prognosis. However, CTCs have some negative characteristics that render CTCs hard to be utilized. They are, for example, scarce and highly heterogeneous. Scientist in this field have longed for breakthrough to overcome these characteristics of CTCs for a long time.

CTCs, as specimen, have strong potential in research. They can be used in basic research, cell expansion, cell function study, cell biology research (e.g., studies of nucleic acids, proteins, and genomics), creation of animal tumor models, and pre-clinical studies including analysis of cancer drug resistance and establishment of clinical correlation (e.g., evaluation of their use in monitoring disease status, assessing treatment effectiveness, and predicting prognosis).

The current sorting techniques can be roughly classified into two categories: physical methods and biomedical methods. The physical methods collect CTCs through their sizes, densities, and dielectrophoretic properties. If compared to the biochemical methods, the physical methods, however, are inferior than the biochemical methods in the matter of sorting accuracy and product purity.

The biochemical methods, on the other hand, filter specimen into CTCs and the other cells by their surface markers. The challenges lie in the biochemical methods are the heterogeneity of CTCs. The heterogeneity includes the differences at the morphological, physiological, and molecular levels. Such differences easily lead to inaccurate results when determining cell counts.

In addition, current techniques are not able to produce satisfactory results if scientists want to study the detailed information about the roles of CTCs in metastasis. The reason for such weakness is that the current techniques are not able to efficiently filter out non-viable CTCs from the entire CTC population. This limitation restricts scientists from identifying and analyzing those CTCs directly amount to metastasis.

For instance, a portion of CTCs is easily lost with the current techniques. The cell diameter of CTCs is usually larger than that of regular blood cells, and several physical methods try to employ such characteristic to isolate CTCs from the others with filters having specific pore size. However, provided that the heterogeneity of CTCs includes the differences in cell diameter, some CTCs with diameters similar to that of leukocytes are also screened out at the same time.

Another example is that a portion of CTCs collected with the current techniques is futile. Based on the short half-life of CTCs, most of the CTCs enter cell death after few hours of circulation in blood stream. It is estimated that only 0.01% of CTCs can metastasize successfully. As that not the entire CTC population has metastatic potential, putting the entire CTC population into CTC count would be misleading.

In order to capture accurate information regarding CTCs and utilize such accurate information in different applications, a technique to effectively isolate and identify CTCs is desired.

### SUMMARY

Some embodiments of the present invention provide methods of assessing circulating tumor cells (CTCs) and methods of using the data obtained from assessing CTCs, as a response to the aforementioned defects of prior arts.

More particularly, the methods of assessing CTCs comprises multiple steps. The first step is providing a specimen, peripheral blood, comprising multiple non-target cells and multiple target cells. The second step is removing a first subset of the multiple non-target cells and sustaining the multiple target cells.

Next, incubating the specimen with a fluorescent dye to label multiple CTCs in the multiple target cells. Then, identifying cells having metastatic potential and cells having metastatic characteristics among the multiple CTCs, and obtaining a biological data from the cells having metastatic potential and from the cells having metastatic characteristics. The last step in these embodiments is applying a first bioassay, selected based on the biological data, to the cells having metastatic potential and the cells having metastatic characteristics.

Methods of using the data obtained from assessing CTCs are also provided. The method comprises a step of providing at least one output obtained from the first bioassay as described in the aforementioned methods, and a step of using the at least one output to determine tumor morphology, monitor tumor status, predict prognosis, provide treatment suggestion, assess treatment effectiveness, or the combination thereof.

The embodiments disclosed above can effectively diminish the negative effects resulted from the heterogeneity of CTCs (e.g., the differences at the morphological, physiological, and molecular levels) on the accuracy of cell counts. The cell counts of CTCs are frequently underestimated because of selection bias, which is also avoided in the embodiments. Moreover, with negative selection, the embodiments can produce higher purity of CTC samples which provide a foundation to more accurate studies for both basic and clinical researches.

There are many inventions described and illustrated herein. The present inventions are neither limited to any single aspect nor embodiment thereof, nor to any combinations and/or permutations of such aspects and/or embodiments. Moreover, each of the aspects of the present inventions, and/or embodiments thereof, may be employed alone or in combination with one or more of the other aspects of the present inventions and/or embodiments thereof. For the sake of brevity, many of those permutations and combinations will not be discussed separately herein.

### BRIEF DESCRIPTION OF THE DRAWINGS

FIG. 1 is a flow chart illustrating a method of assessing circulating tumor cells, in accordance with some embodiments of the present invention.

FIG. 2 is a flow chart illustrating an optional procedure can be performed after the step (a) in FIG. 1, in accordance with one embodiment of the present invention.

FIG. 3 is a flow chart illustrating a detailed procedure expanded from the step (b) in FIG. 1, in accordance with one embodiment of the present invention.

FIG. 4 is a flow chart illustrating a detailed procedure expanded from the step (c) in FIG. 1, in accordance with one embodiment of the present invention.

FIG. 5 is a flow chart illustrating a detailed procedure expanded from the step (d) in FIG. 1, in accordance with one embodiment of the present invention.

FIG. 6 is a flowchart illustrating a method of using the data obtained from assessing circulating tumor cells, in accordance with some embodiments of the present invention.

### DESCRIPTION OF THE PREFERRED EMBODIMENTS

The drawings disclose some preferred embodiments of the present invention, which are intended to be used with the descriptions herein to enable one skilled in the art to understand the claimed features, as well as to make and use the claimed invention.

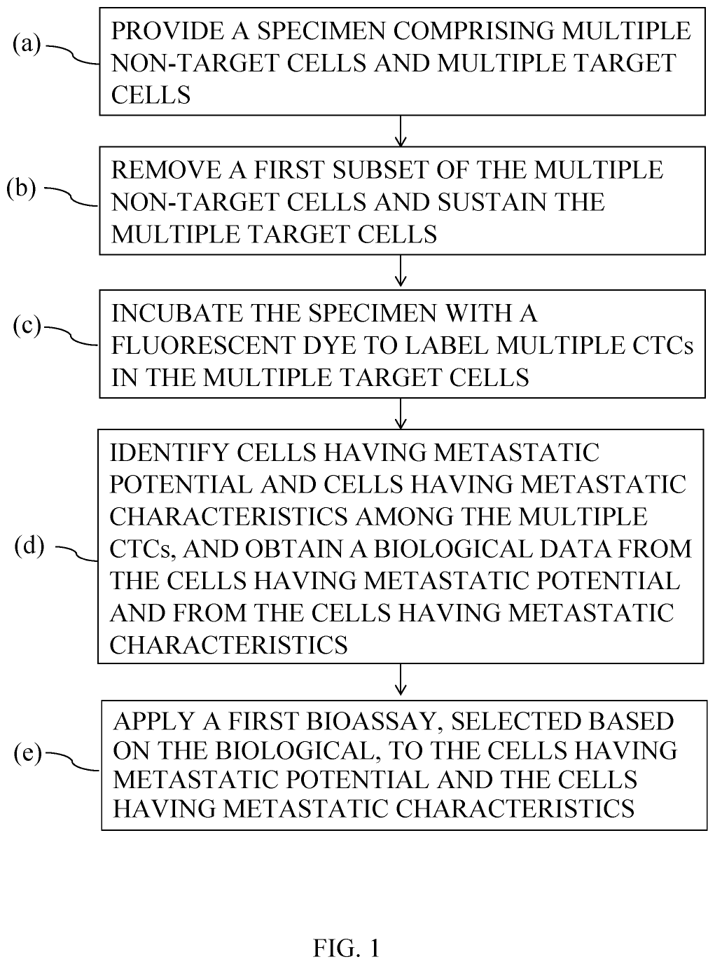

FIG. 1 is a flow chart illustrating a method of assessing circulating tumor cells, in accordance with some embodiments of the present invention. As shown in FIG. 1, the method comprises step (a) to step (e). In step (a), a specimen comprising multiple non-target cells and multiple target cells is provided. Moreover, in step (b), a subset of the multiple non-target cells is removed from the specimen while sustain the live of the multiple target cells.

In the third step, step (c), the specimen is incubated with a fluorescent dye in order to label multiple circulating tumor cells (CTCs) among the multiple target cells. The next step, step (d), is to identify those cells having metastatic potential and those cells having metastatic characteristics in the multiple CTCs, and then obtain a biological data from the cells having metastatic potential and from the cells having metastatic characteristics. At last, step (e) is to perform a first bioassay, selected based on the biological data, to the cells having metastatic potential and the cells having metastatic characteristics. In this embodiment, the specimen is peripheral blood.

Regarding the specimen in step (a), it is preferred that the peripheral blood is obtained from a Homo sapiens who meets the following inclusion criteria:
1) Subjects who are undiagnosed with cancer, newly diagnosed with cancer, or diagnosed with recurrent cancer, and the treatment status of the subjects could be pre-treatment, in treatment, or post-treatment.
2) The cancer is liver cancer, lung cancer, colorectal cancer, breast cancer, nasopharyngeal carcinoma, prostate cancer, esophageal cancer, pancreatic cancer, or head and neck cancers.
3) The cancer is assigned to one from stage 1 to stage 4 based on the 8^{th} Edition AJCC Cancer Staging Manual, in which the tumor can be ether primary or metastatic.

Regarding the peripheral blood in this embodiment, the first 3-5 ml of the peripheral blood is preferred to be discarded to avoid contamination from epithelia cells. The peripheral blood used as specimen is collected in vacutainer tube containing anticoagulants (e.g., tripotassium EDTA) and stored at 4° C. The pre-treatment (step (a) and start the sustaining step in step (b)) is recommended to be completed in the first 6 hours after collection.

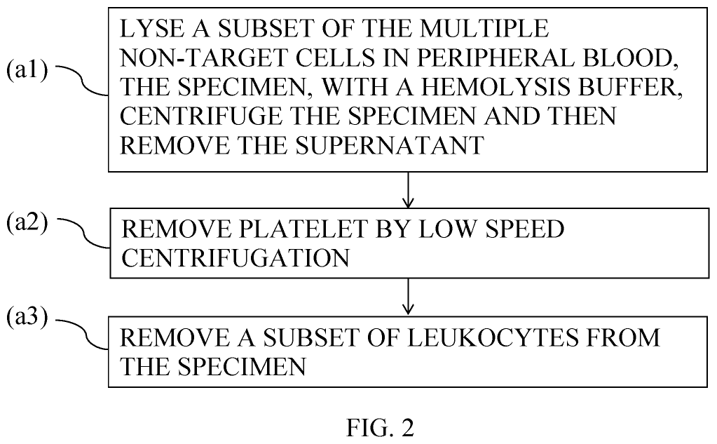

In some extended protocols, the step (a) in the method could be further followed by step (a1) to step (a3). FIG. 2 is a flow chart illustrating an optional procedure that are performed after the step (a) in FIG. 1, in accordance with one embodiment of the present invention. Such step (a1) to step (a3) are used to further remove the non-target cells, and can entirely replace step (b), be used in conjunction with step (b), or be used as additional steps between step (a) and step (b). In one embodiment, the non-target cells are blood cells (including erythrocyte sand leukocytes) or non-viable CTCs, while the target cells are viable CTCs.

The objective of step (a1) is to lyse the multiple non-target cells by incubating the peripheral blood, the specimen, with a hemolysis buffer. The specimen is centrifuged and the supernatant is removed.

In one embodiment, the multiple non-target cells include erythrocytes. Regarding the composition of the hemolysis buffer in the embodiment, 1 L of the hemolysis buffer includes 8.26 g of NH₄Cl, 1.19 NaHCO₃, 200 μl of 0.5M ethylenediaminetetraacetic acid at pH 8. The pH value of the hemolysis buffer is preferred to be at pH 7.3.

In step (a1), the ratio of peripheral blood and hemolysis buffer is 1:10. The incubation of peripheral blood and hemolysis buffer is preferred to be less than 10 minutes. Then centrifuge the mixture and remove the supernatant. After step (a1), the cells are re-suspended with phosphate buffered saline (PBS) and then centrifuged again at low speed to remove platelets.

In step (a3), a subset of leukocytes is removed from the specimen. Most embodiments of the present invention are compatible with commercially available kits used to remove leukocytes. In some embodiments, the leukocytes in the specimen are first labeled with anti-leukocytes antibodies, and bead conjugates against the anti-leukocytes antibodies are then used to capture the anti-leukocytes antibodies and the labeled leukocytes. The multiple target cells not immobilized by the bead conjugates are isolated accordingly. More particularly, the anti-leukocyte antibodies are anti-CD45 antibody in these embodiments.

The objective of step (a3) is to remove a subset of leukocyte from the specimen. In the above embodiments, most of the leukocytes in the specimen are removed by the immunomagnetic bead-based negative selection.

The complete the scenario of immunomagnetic bead-based negative selection is that, after the step (a2), the leukocytes in the specimen are labeled with anti-CD45 antibody, captured by bead conjugates against anti-CD45 antibody, and immobilized by a magnetic field while the other multiple target cells not labeled by the anti-CD45 antibody can freely move and be isolated in the step (a3). This approach can effectively remove most of the leukocytes in the specimen. Even though not all leukocytes are removed in the step (a3), this approach offers the flexibility to choose type and quantity of leukocytes aimed to be removed from the specimen. The choice is determined according the needs and not limited in the present invention.

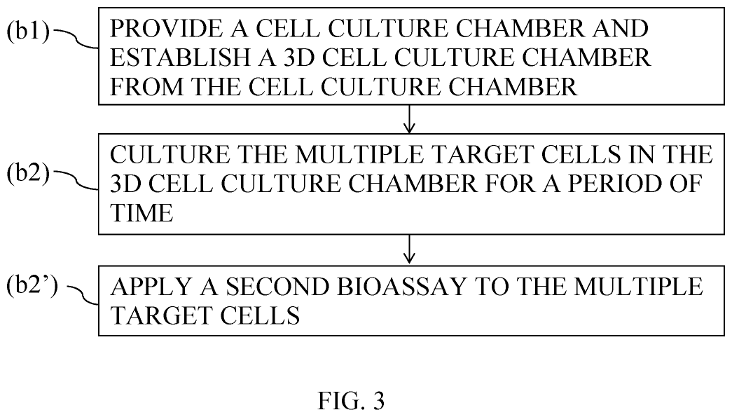

In different embodiments of the present invention, step (a1) to step (a3) may be combined with step (b) or entirely skipped to step (b). The objective of step (b) is to remove the multiple non-target cells and sustain the live of the multiple target cells. FIG. 3 is a flow chart illustrating a detailed procedure expanded from the step (b) in FIG. 1, in accordance with one embodiment of the present invention. The step (b) in this embodiment comprises step (b1) and step (b2), and an optional step (b2′) as well.

In step (b1), a cell culture chamber is provided and used to established a three-dimensional (3D) cell culture chamber. More particularly, the 3D cell culture chamber is established by plating a hydrophilic gel evenly in the cell culture chamber to form a thin layer. The preferred 3D cell culture chamber is based on 3D spheroid cell culture in this embodiment.

In step (b2), the multiple target cells are culture in the 3D cell culture chamber for a period of time. In the embodiment, the period of time is 8 days. In the 3D cell culture chamber, the medium contains epidermal growth factor (EGF), fibroblast growth factor (FGF), and nutrient additives.

After 8 days, a second bioassay may be applied to the multiple target cells. However, this step (b2′) is optional. In this embodiment, the second bioassay is a genomic analysis focusing oncogenes. Nucleic acids are extracted from the multiple target cells in the cell culture chamber after 8 days of incubation. The nucleic acids are, but not limited to, one of ribonucleic acids (RNAs), deoxyribonucleic acids (DNAs), and the combination thereof.

In the embodiment, the nucleic acids are RNAs extracted with PicoPure™ RNA isolation kit.

Then, the nucleic acids are used as the sample to obtain a data set. More particularly, the data set is obtained by analyzing at least one target region on the nucleic acids. Provided that this embodiment uses total RNA as the primary sample, the total RNA needed to be reverse transcribed into complementary DNA (cDNA) before analyzing at least one target gene (e.g., an oncogene) with a real-time polymerase chain reaction (RT-PCR) system. In the embodiment, the at least one target gene may be, but not limited to, ALDH1, CDH1, CDH2, JUP, KRT19, MRP1, MRP2, MRP4, MRP5, MRP7, NANOG, OCT3/4, PROM1, SNAI1, SOX2, TWIST1, VIM, or the combination thereof.

In the same embodiment, housekeeping genes, such as the B2M gene, are used as internal controls to evaluate the relative expression level of the at least one target gene. If the relative expression level of a target gene in a specific specimen is higher than or equal to the median of the relative expression levels of the same gene in all assayed specimens, it is sorted into the high expression group. On the contrary, if the relative expression level of a target gene in a specific specimen is lower than the median of the relative expression levels of the same gene in all assayed specimens, it is sorted into the low expression group.

Although the median of relative expression levels is used to determine which group the target gene belongs to in the above embodiment, it should be known that other cutoff values may be used in other embodiments. For example, one may choose a mean or shift a suitable cutoff value for receiver operating characteristic curve (ROC curve) based on the assay environment or assay requirements.

In step (c), the specimen is incubated with a fluorescent dye to label multiple CTCs in the multiple target cells. More particularly, there may be a second subset of non-target cells or non-viable CTCs present in the pool of target cells. The objective of the step (c) is to identify the viable CTCs.

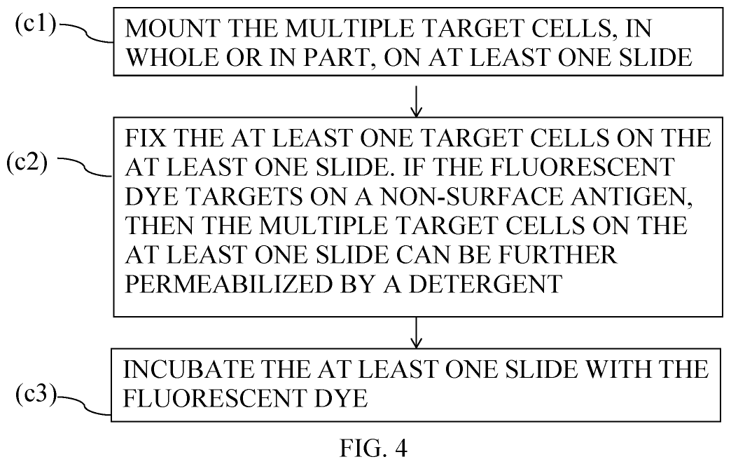

FIG. 4 is a flow chart illustrating a detailed procedure expanded from the step (c) in FIG. 1, in accordance with one embodiment of the present invention. The step (c) is expanded into step (c1) to step (c3) in this embodiment.

In step (c1), the multiple target cells, in whole or in part, is mounted on at least one slide. The actual number of the at least one slide needed in this step can be determined based on the number of bioassay in the following steps. For example, as both cells having metastatic potential and cells having metastatic characteristics are needed to be identified in this embodiment, two slides should be made for these two immunofluorescent assays accordingly. However, one should note that the present invention is not limited by this embodiment.

In step (c2), the multiple target cells are fixed onto the at least one slide. If an immunofluorescent antibody against non-surface antigens is used in the following steps, the multiple target cells on the at least one slide can be further permeabilized by a detergent. In this embodiment, the multiple cells are fixed on the at least one slide by formalin and permeabilized by C₁₄H₂₂O(C₂H₄O)ₙ (Trinton X-100). The permeabilization is required because the antigen for the antibody used here is a non-surface antigen. In some other embodiments where the antigens and techniques are different from this case, the permeabilization could be modified accordingly. Again, one should note that the present invention is not limited by this embodiment.

In step (c3), the at least one slide is incubated with fluorescent dye after the permeabilization. Such fluorescent dye is not limited to any specific primary antibody or secondary antibody. One should choose the antibody based on the nature of the selected antigen.

In this embodiment, with the fluorescent dye, viable CTCs can be identified from those non-target cells which stay with the viable CTCs. The biomarkers used to identify those non-target cells and non-viable CTCs includes CD4, CD8, CD14, CD11b, CD34, CD45, CD68, and the combination thereof. For those erythrocytes which stay with the viable CTCs, another biomarker, CD235a, can be used to identify them. The objective of step (c3) is to mark the leukocytes, erythrocytes, and non-viable cells which are unwanted but failed to be removed in step (a) and step (b), in case they will be mistreated as CTCs in the following steps.

In this embodiment, antibodies against the cell-specific biomarkers of epithelial CTCs (E-CTCs) and mesenchymal CTCs (M-CTCs) are recommended to be used as the primary antibody to label the multiple CTCs.

The cell-specific biomarkers of E-CTCs and M-CTCs are used here is based on the fact that epithelial-to-mesenchymal transition is a signature of metastasis. Identification of these cells associated with metastasis is important for the following analysis and monitoring.

In this embodiment, data, such as cell counts or genomics, collected from the E-CTCs and M-CTCs can provide more accurate and useful information for studying tumors.

More particularly, the biomarker used to identify E-CTCs may be epithelial cell adhesion molecule (EpCAM), cytokeratins (CKs), E-cadherin, claudin, zonula occludens protein-1 (ZO-1), desmoplakin, mucoprotein MUC-1, β-catenin, syndecan-1, or the combination thereof. Immunofluorescences that against the above biomarkers can be used to label E-CTCs in this embodiment.

With regard to M-CTCs, biomarkers such as zinc finger protein SNAI1 (Snail), zinc finger protein SNAI2 (Slug), matrix metallopeptidases (MMPs), vimentin, fibronectin, α-smooth muscle actin (α-SMA), thrombospondin, plasminogen activator inhibitor-1 (PAI-1), transforming growth factor beta (TGF-β), and the combination thereof are preferred. Immunofluorescences that against the above biomarkers can be used to label M-CTCs in this embodiment.

After the step (c1) to step (c3), the next step, step (d), is to obtain a biological data from the cells having metastatic potential and from the cells having metastatic characteristics. In this embodiment, the biological data is cell counts.

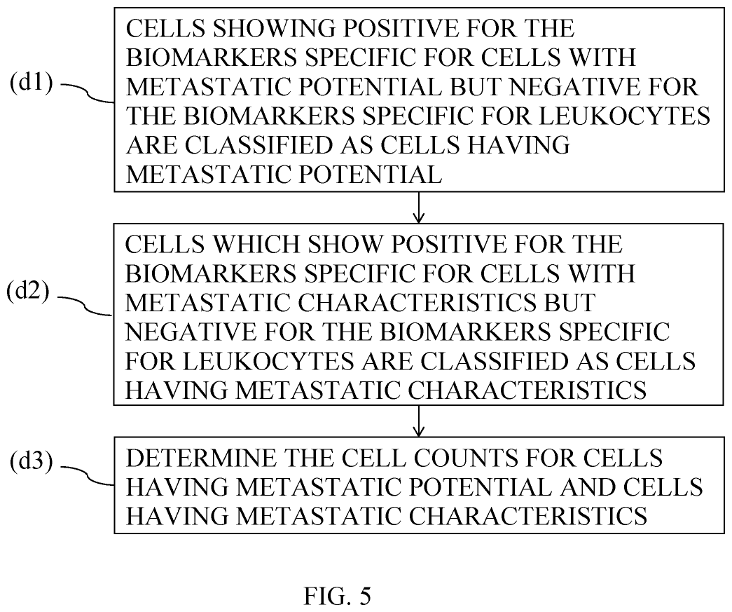

FIG. 5 is a flow chart illustrating a detailed procedure expanded from the step (d) in FIG. 1, in accordance with one embodiment of the present invention. In the embodiment, FIG. 5 comprises of step (d1) to step (d3). The so-called cells having metastatic potential and cells having metastatic characteristics are the M-CTCs and E-CTCs respectively.

In step (d1), cells which show positive for the biomarkers specific for cells with metastatic potential but negative for the biomarkers specific for leukocytes are classified as cells having metastatic potential. Similarly, cells which show positive for the biomarkers specific for cells with metastatic characteristics but negative for the biomarkers specific for leukocytes are classified as cells having metastatic characteristics in step (d2). In step (d3), the cell counts for cells having metastatic potential and cells having metastatic characteristics are determined respectively.

In the last step, step (e), a first bioassay is applied to the cells having metastatic potential and the cells having metastatic characteristics. In the embodiment, the subject-specific data could be added to interpret the result of the first bioassay. The subject-specific data could be, but not limited to, the age, cancer type, cancer stage, treatment received, first evaluation result, life status (i.e., live or dead), or the combination thereof.

In this embodiment, the first bioassay is cell counting with fluorescent microscope. The first bioassay is applied to the cells having metastatic potential and the cells having metastatic characteristics.

Another embodiment introduces biostatistics into the cell counts of E-CTCs and M-CTCs after the cell counting. The cell counts of E-CTCs and M-CTCs undergo survival analysis with Mann-Whitney U test or Cox regression. If p<0.05, it is considered as significant.

Different biostatistics strategies are applied to the cell counts of E-CTCs and M-CTCs, respectively or as a whole, in some other embodiments. However, the present invention is not limited by these embodiments.

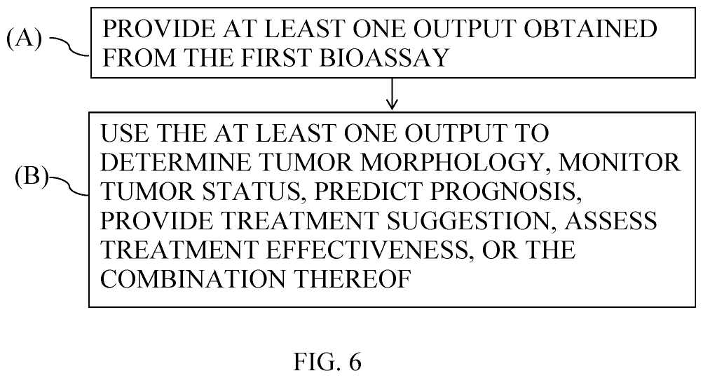

FIG. 6 is a flowchart illustrating a method of using the data obtained from assessing circulating tumor cells, in accordance with some embodiments of the present invention. In the step (A) of FIG. 6, at least one output obtained from the first bioassay in the step (e) is provided. The at least one output is using to determine tumor morphology, monitor tumor status, predict prognosis, provide treatment suggestion, assess treatment effectiveness, or the combination thereof in the next step, step (B).

Based on that the at least one output comes from bioassays applied to the sophistically isolated E-CTCs and M-CTCs, the at least one output is exceptional useful when determining tumor morphology, monitoring tumor status, predicting prognosis, providing treatment suggestion, and assessing treatment effectiveness.

There are many inventions described and illustrated above. The present inventions are neither limited to any single aspect nor embodiment thereof, nor to any combinations and/or permutations of such aspects and/or embodiments. For the sake of brevity, many of those permutations and combinations will not be discussed separately herein.

What is claimed is:

1. A method of assessing circulating tumor cells (CTCs), comprising:
providing a specimen, wherein the specimen is peripheral blood and comprises multiple non-target cells and multiple target cells;
removing a first subset of the multiple non-target cells;
sustaining the multiple target cells;
incubating the specimen with a fluorescent dye to label multiple CTCs in the multiple target cells;
identifying cells having metastatic potential and cells having metastatic characteristics among the multiple CTCs;
obtaining a biological data from the cells having metastatic potential and the cells having metastatic characteristics respectively; and
applying a first bioassay, selected based on the biological data, to the cells having metastatic potential and the cells having metastatic characteristics.

2. The method of assessing circulating tumor cells (CTCs) as claimed in claim 1, wherein in the step of incubating, the biomarker used to identify the second subset of the non-target cells, staying with the multiple CTCs, is one selected from the group consisting of CD4, CD8, CD14, CD11b, CD34, CD45, CD68, CD235a, and the combination thereof.

3. The method of assessing circulating tumor cells (CTCs) as claimed in claim 1, wherein in the step of identifying, the cells having metastatic potential are epithelial circulating tumor cells (E-CTCs) and the cells having metastatic characteristics are mesenchymal circulating tumor cells (M-CTCs).

4. The method of assessing circulating tumor cells (CTCs) as claimed in claim 1, wherein in the step of identifying, the biomarker used to identify the cells with metastatic potential is one selected from the group consisting of epithelial cell adhesion molecule (EpCAM), cytokeratins (CKs), E-cadherin, claudin, zonula occludens protein-1 (ZO-1), desmoplakin, mucoprotein MUC-1, β-catenin, syndecan-1, and the combination thereof.

5. The method of assessing circulating tumor cells (CTCs) as claimed in claim 1, wherein in the step of identifying, the biomarker used to identify the cells with metastatic characteristics is one selected from the group consisting of zinc finger protein SNAI1 (Snail), zinc finger protein SNAI2 (Slug), matrix metallopeptidases (MMPs), vimentin, fibronectin, α-smooth muscle actin (α-SMA), thrombospondin, plasminogen activator inhibitor-1 (PAI-1), transforming growth factor beta (TGF-β), and the combination thereof.

6. The method of assessing circulating tumor cells (CTCs) as claimed in claim 1, wherein the step of sustaining comprises:
providing a cell culture chamber;
establishing a three-dimensional (3D) cell culture chamber from the cell culture chamber;
culturing the multiple target cells in the 3D cell culture chamber for a period of time.

7. The method of assessing circulating tumor cells (CTCs) as claimed in claim 6, wherein the step of sustaining comprises a step of applying a second bioassay to the multiple target cells, and wherein the step of applying is after the step of culturing.

8. The method of analyzing circulating tumor cells (CTCs) as claimed in claim 7, wherein the second bioassay is genomic analysis comprising:
culturing the multiple target cells;
extracting nucleic acids from the multiple target cells; and
obtaining a data set from the nucleic acids.

9. The method of assessing circulating tumor cells (CTCs) as claimed in claim 8, wherein the nucleic acids include ribonucleic acids (RNA), deoxyribonucleic acids (DNA), or the combination thereof.

10. The method of assessing circulating tumor cells (CTCs) as claimed in claim 8, wherein the data set is obtained by analyzing a least one target region on the nucleic acids.

11. The method of assessing circulating tumor cells (CTCs) as claimed in claim 1, wherein the biological data in the step of obtaining are cell counts.

12. The method of assessing circulating tumor cells (CTCs) as claimed in claim 1, wherein the first bioassay in the step of applying is cell count analysis.

13. The method of assessing circulating tumor cells (CTCs) as claimed in claim 12, wherein a subject-specific data is introduced into the first bioassay.

14. A method of using the data obtained from assessing circulating tumor cells (CTCs), comprising:
providing at least one output obtained from the first bioassay of the method of assessing circulating tumor cells (CTCs) as claimed in claim 1;
using the at least one output to determine tumor morphology, monitor tumor status, predict prognosis, provide treatment suggestion, assess treatment effectiveness, or the combination thereof.

15. The method of using the data obtained from assessing circulating tumor cells (CTCs) as claimed in claim 14, wherein in the step of incubating of the method of assessing circulating tumor cells (CTCs), the biomarker used to identify the second subset of the non-target cells, staying with the multiple CTCs, is one selected from the group consisting of CD4, CD8, CD14, CD11b, CD34, CD45, CD68, CD235a, and the combination thereof.

16. The method of using the data obtained from assessing circulating tumor cells (CTCs) as claimed in claim 14, wherein in the step of identifying of the method of assessing circulating tumor cells (CTCs), the cells having metastatic potential are epithelial circulating tumor cells (E-CTCs) and the cells having metastatic characteristics are mesenchymal circulating tumor cells (M-CTCs).

17. The method of using the data obtained from assessing circulating tumor cells (CTCs) as claimed in claim 14, wherein in the step of identifying of the method of assessing circulating tumor cells (CTCs), the biomarker used to identify the cells with metastatic potential is one selected from the group consisting of epithelial cell adhesion molecule (EpCAM), cytokeratins (CKs), E-cadherin, claudin, zonula occludens protein-1 (ZO-1), desmoplakin, mucoprotein MUC-1, β-catenin, syndecan-1, and the combination thereof.

18. The method of using the data obtained from assessing circulating tumor cells (CTCs) as claimed in claim 14, wherein in the step of identifying of the method of assessing circulating tumor cells (CTCs), the biomarker used to identify the cells with metastatic characteristics is one selected from the group consisting of zinc finger protein SNAI1 (Snail), zinc finger protein SNAI2 (Slug), matrix metallopeptidases (MMPs), vimentin, fibronectin, α-smooth muscle actin (α-SMA), thrombospondin, plasminogen activator inhibitor-1 (PAI-1), transforming growth factor beta (TGF-β), and the combination thereof.

19. The method of using the data obtained from assessing circulating tumor cells (CTCs) as claimed in claim 14, wherein the step of sustaining of the method of assessing circulating tumor cells (CTCs) comprises:
providing a cell culture chamber;
establishing a three-dimensional (3D) cell culture chamber from the cell culture chamber;
culturing the multiple target cells in the 3D cell culture chamber for a period of time.

20. The method of using the data obtained from assessing circulating tumor cells (CTCs) as claimed in claim 19, wherein the step of sustaining of the method of assessing circulating tumor cells (CTCs) comprises a step of applying a second bioassay to the multiple target cells, and wherein the step of applying is after the step of culturing.

21. The method of using the data obtained from assessing circulating tumor cells (CTCs) as claimed in claim 20, wherein the second bioassay of the method of assessing circulating tumor cells (CTCs) is genomic analysis comprising:
culturing the multiple target cells;
extracting nucleic acids from the multiple target cells; and
obtaining a data set from the nucleic acids.

22. The method of using the data obtained from assessing circulating tumor cells (CTCs) as claimed in claim 21, wherein the nucleic acids of the method of assessing circulating tumor cells (CTCs) include ribonucleic acids (RNA), deoxyribonucleic acids (DNA), or the combination thereof.

23. The method of using the data obtained from assessing circulating tumor cells (CTCs) as claimed in claim 21, wherein the data set of the method of assessing circulating tumor cells (CTCs) is obtained by analyzing a least one target region on the nucleic acids.

24. The method of using the data obtained from assessing circulating tumor cells (CTCs) as claimed in claim 14, wherein the biological data in the step of obtaining of the method of assessing circulating tumor cells (CTCs) are cell counts.

25. The method of using the data obtained from assessing circulating tumor cells (CTCs) as claimed in claim 14, wherein the first bioassay in the step of applying of the method of assessing circulating tumor cells (CTCs) is cell count analysis.

26. The method of using the data obtained from assessing circulating tumor cells (CTCs) as claimed in claim 25, wherein a subject-specific data of the method of assessing circulating tumor cells (CTCs) is introduced into the first bioassay.
